(19) United States
(12) Patent Application Publication
(10) Pub. No.: US 2006/0217625 A1
(43) Pub. Date: Sep. 28, 2006

(54) Mouthpiece for breath tester

(76) Inventor: Macquorn Rankine Forrester JR., St. Louis, MO (US)

Correspondence Address: JOHN S. BEULICK;C/O ARMSTRONG TEASDALE, LLP, ONE METROPOLITAN SQUARE, SUITE 2600, ST LOUIS, MO, 63102-2740, US

(21) Appl. No.: 11/089,655

(22) Filed: Mar. 25, 2005

(51) Int. Cl. A61B 5/08

(52) U.S. Cl. 600/532

(57) ABSTRACT

A breath tester mouthpiece that includes an increased flexibility section so that breath testing can be performed with a subject in a variety of different positions relative to a tester (e.g., an officer) is provided. The mouthpiece includes an open first end, a closed second end, and a body extending therebetween. The body includes an increased flexibility section and has a substantially D-shaped cross-sectional shape. The body also includes at least one port for channeling air blown into the mouthpiece therethrough.

### BACKGROUND OF THE INVENTION

This invention relates generally to breath testing devices, and more specifically, to a mouthpiece for a breath tester.

Breath testing devices typically are used by police officers when assessing whether a driver has consumed an amount of alcohol in excess of the legal limit in that particular jurisdiction. Police officers have several concerns when performing an alcohol breath test, including safety. For safety reasons, an officer typically would prefer positioning himself directly in front of a subject, with the subject facing the officer at an arm's length. The officer would hold the breath testing device in one hand, i.e., hold the device using only his non-weapon hand outstretched towards the subject, and stand slightly sideways to the subject to gain balance and a low center of gravity. In this position, a potentially belligerent subject is kept at arm's length and at the opposite side of the officer's weapon, so the subject cannot grab the officer or any of his equipment. Also, the officer's other hand is kept free and accessible to his gun or other non-lethal weapons that may be needed.

When in this position, the officer also can keep his face comfortably turned on the subject to observe the subject throughout the entire test. Specifically, there are generally subject-blowing cues on the display that the officer needs to observe while also observing the subject.

Further, during a test, a subject should not be able to view the device display. If the subject can view the display during a test, the subject may try to manipulate the manner of blowing, which may diminish the ability of the officer to get the best breath test result. Also, the subject's discard breath should not be directed at the officer for health reasons. In addition, it would be beneficial to accommodate testing positions in which both the officer and the subject are standing, a sitting subject (e.g., in a driver or passenger seat of an automobile) and a standing officer, and a laying subject, as well as any known orientation in which breath alcohol tests are performed.

Breath testing devices typically include a removable mouthpiece. For health reasons, a new mouthpiece is used for each subject. Since much alcohol breath testing is done at night under less than ideal lighting conditions, the mouthpiece should mount to the housing in an easy and intuitive manner. Traditionally, mouthpieces have holes in the side of them, which are required to align with ports on the instrument housing. One port is provided so that breath is drawn into the internal fuel cell sensor and another port may be used to measure pressure in the mouthpiece for flow measurement of the breath. These ports mate with the holes in the mouthpiece with an airtight seal for proper instrument performance. Before starting a test, a clean mouthpiece must be oriented and lined up so the holes align properly with the ports and then pushed straight on. This is not always easily done under less than ideal lighting conditions. Also, the manner in which the mouthpiece is mounted is not necessarily intuitive and may require the officer's close observation.

With known breath testers, and to maintain the display in view of the officer, the officer typically stands to the side of the subject. In this position, the officer compromises his ability to keep the subject in plain view in front of him and is positioned less than a full arm's length from the subject. The subject also has some opportunity to view the display. If the officer moves away from the subject, this compromises the officer's view of the display and the subject's discard breath may be directed towards the officer.

### BRIEF DESCRIPTION OF THE INVENTION

In one aspect, a breath tester mouthpiece that includes an increased flexibility section so that breath testing can be performed with a subject in a variety of different positions relative to a tester (e.g., an officer) is provided. More specifically, with the mouthpiece described herein, an officer can avoid being blown on by a subject, can simultaneously view the display, the subject and the surrounding environment, can position himself to avoid undo risk (e.g., a weapon hand free as well as positioning weapons away from the subject), can sample the subject at an extended arm's length, can install the mouthpiece without having to look at the instrument, and can be positioned relative to the subject in all known orientations in which breath alcohol tests are performed (e.g., the subject standing, sitting, or laying). No known mouthpiece provides this combination of advantages.

More particularly, and in one embodiment, the mouthpiece includes an open first end, a closed second end, and a body extending therebetween. The body includes an increased flexibility section and has a substantially D-shaped cross-sectional shape. The body also includes at least one port for channeling air blown into the mouthpiece therethrough.

As explained above, the mouthpiece enables a breath test to be conducted with a subject being in a variety of different positions relative to a tester (e.g., an officer). For example, an officer can stand directly in front of the subject without being blown on while still being able to view an instrument display and surrounding environment. In addition, a subject can be sitting in a vehicle with an officer standing outside the vehicle, and a breath test can be conducted without the subject having to contort himself/herself in order to provide a breath sample. Other orientations are possible and contemplated.

### BRIEF DESCRIPTION OF THE DRAWINGS

FIG. 1 is a front perspective view of an example hand-held breath tester;

FIG. 2 is a perspective top view of a mouthpiece that can be used in connection with the breath tester shown in FIG. 1;

FIG. 3 is a side cross sectional view of the mouthpiece shown in FIG. 2; and

FIG. 4 is a perspective bottom view of the mouthpiece shown in FIG. 2.

### DETAILED DESCRIPTION OF THE INVENTION

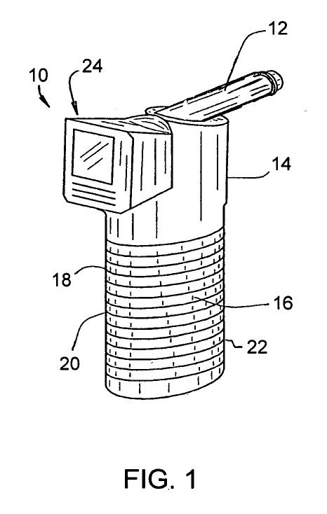

An exemplary embodiment of a hand-held breath tester is shown in FIG. 1. Tester 10 includes a mouthpiece 12 and a housing 14 having a pair of opposed sidewalls 16,18 that are connected together along a front edge 20 and an axially-spaced rear edge 22, such that a cavity is defined therebetween. Electronic components are housed within housing 14.

Housing 14 also includes at least one actuator (not shown) and a display 24. More specifically, housing 14 includes a light illumination actuator and a manual sample actuator. The actuators, in the example embodiment, are depressible buttons. The manual sample actuator is centered along housing rear edge 22, and display 24 is centered opposite the sample actuator and along front edge 20. Depressing the manual sample actuator enables an operator performing a breath test to take a manual sample, rather than an automatic sample. Display 24 enables the results of breath testing to be visually displayed to the operator during the breath testing. Depressing the light illumination actuator causes display to be internally illuminated during a breath test.

Breath tester is illustrated and described herein by way of example only. The mouthpiece described below can be used in connection with many different types of testers and is not limited to practice with any one particular tester.

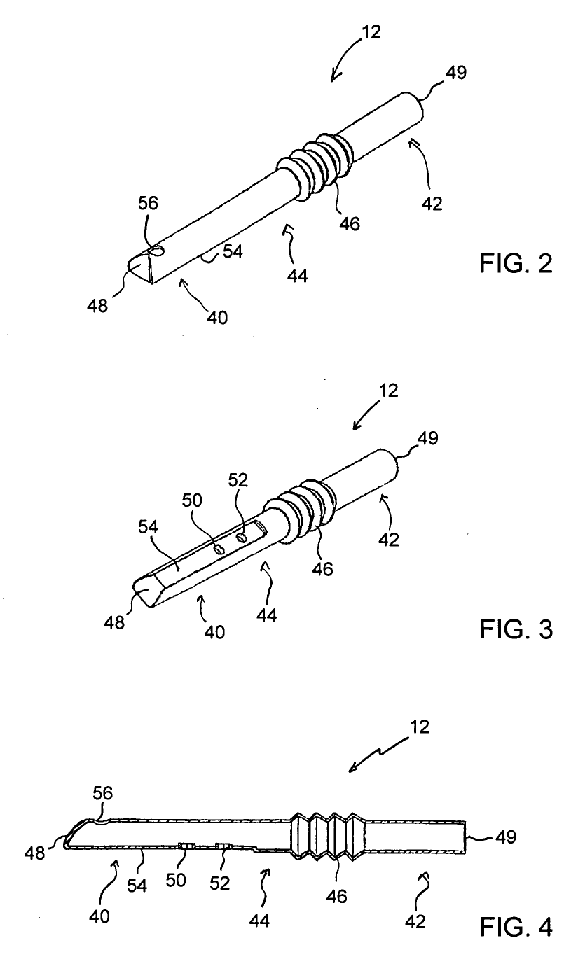

FIG. 2 is a perspective view of an example embodiment of mouthpiece 12. FIG. 3 is a side cross sectional view, and FIG. 4 is a perspective bottom view of mouthpiece 12. Referring to FIGS. 2, 3 and 4, and in the exemplary embodiment, mouthpiece 12 is a hollow tube that includes coupling portion 40, an insertion portion 42, and a substantially cylindrical portion 44 extending therebetween. Mouthpiece 12 also includes an increased flexibility section 46 which facilitates desired positioning of a tester and an operator relative to a subject being tested. The term “increased flexibility” refers to the relative flexibility of section 46 compared to another section of mouthpiece 12, and increased flexibility section 46 facilitates bending and extending mouthpiece 12 so that a test subject and a tester (e.g., an officer) can be positioned as desired by the tester. It is contemplated, for example, the a mouthpiece could include more than one increased flexibility section, and the increased flexibility section shown in FIGS. 2, 3 and 4 is by way of example only.

Mouthpiece 12 may be any configuration or device, and is not limited to being a tubular configuration, that facilitates channeling a subject's breath into the tester housing. As described above, coupling portion 40 provides that mouthpiece 12, as described in more detail below, can be fully inserted within interface of a tester housing. Coupling portion 40 can be of many different configurations depending on the particular tester housing being utilized.

Increased flexibility portion 46 enables testing from any one of numerous different positions. For example, an officer may be positioned directly in front of a subject without being blown on while still being able to view instrument display 24 and the surrounding environment. This is a common way of testing when both the officer and the subject are out of the car and standing at roadside. In addition, a driver or passenger can be tested when the subject is in the car and the officer is performing a test through the window. This type of testing occurs in roadblocks, and can be performed without the officer having to contort himself. The sample can be collected and the officer can view the display while the breath flow is directed away from the officer. Testing can also occur with the subject in other orientations for a variety of reasons. These reasons include but are not limited to the operator being taller than the subject, the operator being shorter than the subject, or even when the subject is laying on the ground. Increased flexibility section 46 also facilitates the packaging and storage of mouthpieces 12 in a straight orientation, which is more efficient and easier than having to package and store mouthpieces with other orientations.

Increased flexibility section 46 is formed in the example embodiment by an “accordion” shaped section that enables bending and extension of mouthpiece 12. It is contemplated that section 46 can be formed by other shapes that facilitate such bending and extension.

Insertion portion 42 provides an airflow passage and is configured to be inserted in a person's mouth being tested. A stop may be provided to limit the extent to which insertion portion 42 can be inserted within the person's mouth. In the example embodiment, insertion portion 42 has a substantially circular cross-sectional profile. It should be noted that mouthpiece insertion portion 42 is not limited to having a substantially circular cross-sectional profile, but rather, insertion portion 42 may be any shape or configuration that facilitates channeling air from the subject towards coupling portion 40, such as, but not limited to a reed-like configuration, and/or a non-circular cross-sectional profile.

Coupling portion 40 is also hollow and extends from body portion 44 to a radially inner end 48. Inner end 48 is rounded and is sealed such that airflow entering inlet end 49 is channeled through a pair of ports 50 and 52 and into the breath testing housing. Coupling portion 40 has a cross-sectional profile that substantially mirrors at least a portion of the cross-sectional profile defined within at least a portion of the housing channel in which mouthpiece 12 is inserted. As such, coupling portion 40 is configured to facilitate mouthpiece 12 being received only in the proper orientation with respect to the tester housing. In the example embodiment, coupling portion 40 includes a radially inner surface 54 that is substantially planar, such that in the exemplary embodiment, coupling portion 40 has a substantially D-shaped cross-sectional profile. It should be noted that coupling portion 40 is not limited to having a substantially D-shaped cross-sectional profile, but rather, coupling portion 40 may have cross-sectional shape that enables coupling portion 40 to function as described herein, such as, but not limited to a substantially circular cross-sectional profile, and/or a frusto-conical cross-sectional profile. Specifically, the combination of the cross-sectional profiles of coupling portion 40 and the channel in the housing in which the mouthpiece is received, and the substantially mating contours of coupling portion 40 and the housing channel bottom surface, facilitates sealing contact being maintained between coupling portion 40 and the channel bottom surface when mouthpiece 12 is coupled to the housing. In an alternative embodiment, mouthpiece 12 and the channel bottom surface are in contact such that sealing contact is created between sidewalls defining the housing ports and mouthpiece ports 50 and 52, respectively.

Coupling portion 40 also includes a discard breath port 56 that discharges discarded breath from the breath testing device. More specifically, and as described in more detail below, the combination of the housing channel and coupling portion 40 enables mouthpiece 12 to be oriented such that the test subject's discarded breath is discharged through port 56 but not towards the operator performing the breath testing. In an alternative embodiment, discard breath port 56 could be located, but is not limited to being located, anywhere along a side, top, or bottom of mouthpiece 12.

During use, initially a detachable mouthpiece 12 is coupled to the housing 14. The method of mouthpiece insertion is intuitive to the operator without the use of a manifold and clearly snaps into a “home” or “testing” position with no ambiguity. The instrument mouthpiece receiver is dead-ended, and the mouthpiece 12 itself has blunt closed end 48, which is simply placed against the dead end of the housing receiver. Because the housing channel is rounded, as is mouthpiece end 48, mouthpiece 12, although D-shaped in cross section, can only be pivotally coupled within the channel in one orientation with respect to the housing. More specifically, because mouthpiece end 48 is rounded or “toe-shaped”, once mouthpiece 12 is coupled within the receiver, mouthpiece 12 can be rotated downward without end 48 coming out of the channel. In other words, once mouthpiece end 48 is coupled within the receiver and adjacent the dead end, mouthpiece end 48 is trapped in a loose, but effective pivot.

Mouthpiece 12 is then pivoted downward towards the housing channel, such that ports 50 and 52 are aligned with, and engage the housing channel ports as mouthpiece 12 is coupled into the housing channel. More specifically, although the housing ports mate with the mouthpiece ports 50 and 52 at an angle (due to the pivoting action), the substantially planar bottom surfaces of mouthpiece 12 and the channel facilitates sealing between the housing ports and ports 50 and 52. Moreover, since the mouthpiece 12 has a D-shaped cross-sectional profile, orientation is intuitive. More specifically, in the exemplary embodiment, because the housing channel sidewalls are rounded past center, when mouthpiece 12 nears the end of its pivoting motion, it forces the housing sidewalls outward, working against the spring force of the molded plastic sidewalls, and then snaps within the channel 44. In an alternative embodiment, the housing sidewalls are substantially rigid and mouthpiece insertion portion 42 is flexible and deformable during insertion of mouthpiece 12 within the housing channel.

When the mouthpiece 12 is in the subject's mouth, the mouthpiece 12 exits the mouth at a right angle to the plane of the subject's face, and as such the display 24 is tipped up towards the eyes of the officer. Thus, when the officer holds assembly 10 in the natural and intuitive manner in the subject's mouth, the officer is easily able to keep the subject in plain view while comfortably monitoring display 24 at the same time. Moreover, because housing 14 includes a display 24 and actuators that are along the same axis, display 24 and actuators are also aligned with the operator's or officer's view while holding assembly 10 in the intuitive and natural position. Moreover, unlike other known devices, display 24 and the actuators are on the edges of the assembly. Accordingly, when the assembly is held in the natural and intuitive manner in an outstretched arm, display 24 is directly in the line-of-sight of the officer, and mouthpiece 12 points to the subject, thus accommodating the officer's need to be in the preferred position while administering the breath test. More specifically, the display 24 and the subject being tested are both in line and in his view, one in front of the other. As such, the subject cannot see the display 24, and the breath discharged from the mouthpiece 12 is channeled upward through discard breath port 56, such that the subject's discard breath is not directed at the officer.

In addition, the cross-sectional shape of mouthpiece 12 enables mouthpiece 12 to only mount one way in an intuitive manner, whether the housing is grabbed with the operator's left or right hand. Accordingly, the natural and intuitive way to hold the assembly in either case is with the arm outstretched, mouthpiece 12 pointed toward the subject, and display 24 pointed towards the officer and tipped up towards the officer's eyes.

When testing is completed, mouthpiece 12 is removed and discarded. To facilitate removal of the mouthpiece 12 from housing 14, in one embodiment, a mouthpiece ejector can be utilized to displace the mouthpiece from the home position. For example, the mouthpiece ejector could be, but is not limited to being, spring loaded or biased with a release, that is selectively operable either manually, or by depressing an actuator.

In the exemplary embodiment, because mouthpiece 12 extends outwardly from the housing, mouthpiece inlet end 49 may be tapped against a generally solid surface, such as, for example, a roof of a car, a table, or a leg of the tester's leg, to forcibly eject mouthpiece 12 from the housing without requiring an operator to physically touch the unsanitary mouthpiece inlet end 49. More specifically, when such an action is taken, mouthpiece 12 rotates out of position in a rotational direction that is opposite the direction of rotation of mouthpiece 12 during insertion of mouthpiece 12 within the housing, without inducing any undue pressure to the ports, and without damaging the housing.

There are a variety of ways to shape a mouthpiece such that it has a blunt, closed end that is easily oriented in a pivot, such that its movement is angular as it approaches the instrument ports, has a substantially planar surface around the mating holes, and snaps into the “home” position. Accordingly, the mouthpiece could be partially V-shaped in cross section for orientation during insertion into the pivot as well as to facilitate movement past the side walls of the mouthpiece receiver. Moreover, there could be a substantially planar area at the bottom of the “V” in order to facilitate sealing on the ports. In such an embodiment, the receiver walls may not extend arcuately from the receiver bottom surface.

In addition, although in the described embodiment, the angular movement of the mouthpiece is in a substantially vertical plane when the instrument is in the upright position, in alternative embodiments, the mouthpiece could be moved in a horizontal plane or at some other angular orientation as well. Furthermore, instead of the “toe” of the mouthpiece being the direct pivot point, alternate pivot points could be formed on the sides of the mouthpiece, and/or fitted to a shape on the receiver, in order to get the same type of angular mouthpiece movement.

Furthermore, although the mouthpiece is described has having only two ports, in alternative embodiments, the mouthpiece could have more or less than two ports depending on the desired application. Moreover, the second port does not have to be a pressure port, but rather the second port could be used to allow air to flow to a thermistor mounted to a pin that extends into the mouthpiece to measure flow, while remaining sealed around the pin. In addition, there could be similar ports for measuring breath temperature or some other relevant phenomenon.

While the invention has been described in terms of various specific embodiments, those skilled in the art will recognize that the invention can be practiced with modification within the spirit and scope of the claims.

What is claimed is:

1. A mouthpiece for a breath testing device, said mouthpiece comprising a body comprising a first end, a second end, said first end being open so that a subject can blow air into said mouthpiece, said second end being closed, said mouthpiece further comprising at least one port for channeling air blown into said mouthpiece into the breath testing device, said mouthpiece further comprising an increased flexibility section.

2. A mouthpiece in accordance with claim 1 wherein said mouthpiece further comprises a discard breath outlet oriented such that discard breath is not directed at an operator of the breath testing device during testing.

3. A mouthpiece in accordance with claim 1 wherein said mouthpiece snaps into engagement with the breath testing device.

4. A mouthpiece in accordance with claim 1 further comprising a stop extending radially outward from said body to facilitate positioning a subject's mouth during breath testing.

5. A mouthpiece for a breath tester, said mouthpiece body comprising a substantially planar surface and an increased flexibility section.

6. A mouthpiece in accordance with claim 5 wherein a portion of said mouthpiece has a selected cross-sectional shape, said selected cross-sectional shape being one of: a D-shaped cross-sectional shape and a V-shaped cross-sectional shape.

7. A mouthpiece in accordance with claim 5 wherein said body further comprises at least one port for channeling air therethrough into the breath testing device.

8. A mouthpiece in accordance with claim 5 wherein said body further comprises at least a first port for channeling for air flow into the breath testing device for sampling, and a second port for channeling air flow to a sensor.

9. A mouthpiece in accordance with claim 5 wherein said body further comprises at least a first port for channeling for air flow into the breath testing device for sampling, and a second port for channeling air flow to at least one of a pressure sensor and a thermistor.

10. A mouthpiece in accordance with claim 5 wherein said body further comprises a plurality of ports for channeling air flow to a differential pressure measuring sensor.

11. A breath tester housing assembly comprising:
a housing comprising a base, a display, and a mouthpiece interface, said base to be gripped by an operator during testing, said display oriented with respect to said housing to be in line with an operator's direct line of view while gripping said base; and
a mouthpiece configured to be removably coupled to said mouthpiece interface, said mouthpiece comprising a body comprising at least one substantially planar surface, said mouthpiece further comprising an increased flexibility section.

12. A housing assembly in accordance with claim 11 wherein said housing further comprises at least one actuator for controlling operation of the breath tester.

13. A housing assembly in accordance with claim 12 wherein said at least one actuator comprises a manual sample located on an edge of said housing base that is opposite an edge where said display is located, such that when an operator stands with the subject and said display in the operator's direct field of view, the operator may at any time press said manual sample without compromising the operator's grip on said base.

14. A housing assembly in accordance with claim 13 wherein said housing comprises a first sidewall and an opposite second sidewall coupled together at a front edge and a back edge, said first and second sidewalls extending radially between a top surface and a bottom surface, said display located along said front edge, said mouthpiece interface located along said top surface.

15. A housing in accordance with claim 14 wherein said mouthpiece interface is oriented with respect to said housing such that said mouthpiece extends outward from said housing back edge when said mouthpiece is coupled to said housing.

16. A breath testing device mouthpiece, said mouthpiece comprising a first end, a second end, and a body extending therebetween, a portion of said body has a selected cross-sectional shape, said selected cross-sectional shape being one of: a D-shaped cross-sectional shape and a V-shaped cross-sectional shape, said body further comprising a passageway extending through said body from said first end towards said second end, said passageway for channeling air blown into said mouthpiece into the breath testing device, said mouthpiece further comprising an increased flexibility section.

17. A mouthpiece in accordance with claim 16 wherein said body further comprises an external surface, an internal surface, and at least one inlet port extending therebetween, said inlet port for channeling air from the passageway into the breath testing device.

18. A mouthpiece in accordance with claim 16 wherein said body further comprises an external surface, an internal surface, and at least one outlet port extending therebetween, said outlet port for channeling discard breath air from the mouthpiece during testing.

19. A mouthpiece in accordance with claim 18 wherein said at least one outlet port is oriented with respect to said mouthpiece such that discard breath is not directed at an operator of the breath testing device during testing.

20. A mouthpiece in accordance with claim 16 wherein at least a portion of said body comprises a substantially planar surface.

21. A mouthpiece in accordance with claim 20 wherein said body further comprises an external surface, an internal surface, and at least one port extending therebetween, said internal surface defining said passageway, said at least one port extends through said substantially planar surface.

22. A mouthpiece in accordance with claim 16 wherein one of said first end and said second end is rounded to facilitate engagement with the breath testing device.

23. A mouthpiece for a breath testing device, said mouthpiece comprising a body extending between a first end and a second end, said body comprising a first body portion, a second body portion, and a passageway defined at least partially within said first and second body portions, said first body portion extending from said first end to said second body portion, said second body portion extending from said second end to said first body portion, said passageway substantially concentrically aligned with respect to said body and extending from said first end towards said second end for channeling air blown into said first end into the breath tester, at least one of said first and second body portions comprising at least one port extending between an external surface of said body and said passageway, and an increased flexibility section.

24. A mouthpiece in accordance with claim 23 wherein said at least one port is defined within said second body portion for channeling air blown into said first end into the breath testing device during testing.

25. A mouthpiece in accordance with claim 23 wherein said at least one port is defined within said first body portion for channeling discard breath air from said mouthpiece during testing, said at least one port is oriented with respect to said mouthpiece such that discard breath is not directed towards an operator of the breath testing device during testing.

26. A mouthpiece in accordance with claim 23 wherein said second end is closed, said first end is open to enable a subject being tested to blow air into said mouthpiece.

27. A mouthpiece in accordance with claim 26 wherein said second end is rounded to facilitate engagement with the breath testing device.

28. A mouthpiece in accordance with claim 26 wherein one of said first body portion and said second body portion has a selected cross-sectional shape, said selected cross-sectional shape being one of: a D-shaped cross-sectional shape and a V-shaped cross-sectional shape.

29. A mouthpiece in accordance with claim 26 wherein one of said first body portion and said second body portion has a substantially semi-circular cross-sectional shape.
